(19) United States
(12) Patent Application Publication
(10) Pub. No.: US 2020/0289715 A1
(43) Pub. Date: Sep. 17, 2020

(54) BIOCOMPATIBLE SOFT TISSUE IMPLANT

(71) Applicant: Carl Freudenberg KG, Weinheim (DE)

(72) Inventors: Karl-Heinz Heffels, Pfungstadt (DE); Denis Reibel, Herrlisheim (FR); Dirk Grafahrend, Mannheim (DE); Guenter Germann, Heidelberg (DE); Oliver Boegershausen, Dieburg (DE)

(21) Appl. No.: 16/758,858

(22) PCT Filed: Oct. 26, 2018
(86) PCT No.: PCT/EP2018/079407

§ 371 Date: Apr. 24, 2020

(30) Foreign Application Priority Data
Oct. 26, 2017 (DE) 10 2017 009 989.8

(51) Int. Cl. A61L 27/58; A61L 27/56; A61L 27/22; A61L 27/24; A61L 27/34; A61L 27/18; A61L 27/54; A61L 27/48

(52) U.S. Cl. CPC A61L 27/58; A61L 27/56; A61L 27/222; A61L 27/24; A61L 27/227; A61L 2430/34; A61L 27/18; A61L 27/54; A61L 27/48; A61L 2400/18; A61L 2420/02; A61L 27/34

(57) ABSTRACT

A biocompatible soft tissue implant for introduction into a human body includes at least one layer comprising an elastomeric material, and at least one textile fabric arranged on the at least one layer comprising the elastomeric material. The at least one textile fabric forms a surface of the biocompatible soft tissue implant. The at least one textile fabric includes bioresorbable fibers which are embedded at least partially in the at least one layer comprising the elastomeric material.

### CROSS-REFERENCE TO PRIOR APPLICATIONS

This application is a U.S. National Phase Application under 35 U.S.C. § 371 of International Application No. PCT/EP2018/079407, filed on Oct. 26, 2018, and claims benefit to German Patent Application No. DE 10 2017 009 989.8, filed on Oct. 26, 2017. The International Application was published in German on May 2, 2019 as WO 2019/081708 under PCT Article 21(2).

### FIELD

The present invention relates to a soft tissue implant for implantation into a human body. The invention also relates to a method for producing the soft tissue implant.

### BACKGROUND

Elevated requirements, for example good biocompatibility, are placed on materials which are to be introduced into a human body. Biocompatibility refers to the property of materials in a biological environment to perform their predetermined functions adapted to the situation, while the host body simultaneously acceptably reacts to the material. The biocompatibility of medical products is checked within the scope of their approval according to the DIN EN ISO 10993 standard. Hereinafter, we understand “biocompatible” materials to be those which have passed the test according to DIN EN ISO 10993 (year).

In this respect, particularly elevated requirements are placed on materials which are intended to remain permanently in the human body as implants. implant is a material which is implanted into the body and is intended to remain permanently or for a period of a few days up to 10 years in the human body. Medical implants have the function of supporting or replacing body functions, while in the case of plastic implants, the shape of body parts which may be destroyed is to be restored or changed. For example, breast implants as implants for soft tissue are used for breast reconstruction or for breast enlargement. Further applications for such soft tissue implants are prostheses for calf muscles or cheek, nose, buttocks, testis or brachialis muscle implants.

Although the silicone often used in implants is basically biocompatible, undesired immune reactions nevertheless occasionally occur. The host body's immune system is activated after implantation and attempts to resorb the foreign material. If the immune cells do not achieve resorption on account of the foreign material properties, the body begins to encircle the implant with a fiber-containing shell and to thereby separate it from the surrounding tissue. This separation becomes alarming at least when the capsule of scar tissue hardens and leads to deformations of the surrounding tissue.

It is known that the surface and structure of an implant are critical with respect to the host body's handling of the implant. Structured surfaces exhibit higher acceptance in the host bodies with less occurrence of the capsule formations described above. (US 2012/0209381, structured surface less capsule contraction) The typically used structured materials are disadvantageous in that they do not permit direct interaction of endogenous tissue with the implant, such that they are not 100% fixed at the implantation site.

### SUMMARY

In an embodiment, the present invention provides a biocompatible soft tissue implant for introduction into a human body. The biocompatible soft tissue implant includes at least one layer comprising an elastomeric material, and at least one textile fabric arranged on the at least one layer comprising the elastomeric material. The at least one textile fabric forms a surface of the biocompatible soft tissue implant. The at least one textile fabric includes bioresorbable fibers which are embedded at least partially in the at least one layer comprising the elastomeric material.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1: Result of tensile test of pure silicone layer as a reference.

FIG. 2: Result of tensile test of the soft tissue implant of Example 1.

FIG. 3: Microscopic image of the surface of the soft tissue implant of Example 1 after two weeks of storage at 37° C. in PBS.

FIG. 4: Schematic cross-section of a soft tissue implant according to an embodiment of the invention.

FIG. 5: Schematic cross-section of a soft tissue implant according to an embodiment of the invention as a breast implant.

FIG. 6: Microscopic image of the sectional view of a soft tissue implant according to an embodiment of the invention.

### DETAILED DESCRIPTION

One approach is to employ biocompatible materials for the surface of implants which can interact with the host body. Such materials can be bioresorbable materials which can be decomposed and metabolized or excreted by endogenous cells. If such materials are designed as support structures, cells can migrate into such structures in order to construct new endogenous tissue. The support structure material is resorbed in the meantime (U.S. Pat. No. 6,638,308 B2 Bioresorbable breast implant) (WO 96/18424: Breast Tissue Engineering).

There are currently no products on the market that follow this approach. Presumably, this is because the implants would lose their function in the course of resorption.

Starting therefrom, an embodiment of the present invention provides a soft tissue implant, in particular a breast implant, for introduction into a human body, which implant at least partially overcomes the aforementioned disadvantages and is well-accepted by the immune system when introduced into the human body and has good long-term stability.

According to an embodiment, the present invention provides a biocompatible soft tissue implant, in particular a breast implant, for introduction into a human body, comprising at least one layer comprising an elastomeric material, and at least one textile fabric arranged on such layer and forming the surface of the soft tissue implant, wherein the textile fabric has bioresorbable fibers which are at least partially embedded in the layer of elastomeric material.

In the soft tissue implant according to an embodiment of the invention, the connection between textile fabric and elastomeric material can be mediated via the bioresorbable fibers, which are at least partially embedded into the layer of elastomeric material.

This embedding can be obtained, for example, by applying the textile fabric to an elastomeric precursor material, for example an unvulcanized silicone layer, and pressing it into the latter. Pressing has the purpose of introducing the fibers of the textile fabric into the precursor layer. The composite can subsequently be solidified, for example by vulcanization of the precursor to form the elastomeric material, and hardened in its elastomeric portion.

By embedding the bioresorbable fibers into the layer of elastomeric material, a stable composite material with high layer adhesion can be obtained. A high layer adhesion means that the soft tissue implant can be handled in the usual way and can be introduced into the human body, for example, without the adhesion between elastomeric material and textile fabric detaching.

Moreover, the soft tissue implant according to the invention offers a plurality of further advantages when introduced into a human body.

For example, the soft tissue implant is advantageous in that it has a surface which is formed from a textile fabric having bioresorbable fibers, since this allows a biocompatibility-increasing interaction with the surrounding tissue. Due to their fiber structure, textile fabrics have a three-dimensional structuring. As explained above, structured surfaces can minimize the frequency of occurrence of unwanted immune responses, such that such a surface is outstandingly suitable for implants and other medical products that interact with the body as a biological system. Non-woven fabrics are particularly preferred since the fibers therein are arranged randomly and have a strong three-dimensional structuring.

A possible measure for the characteristic of the three-dimensional structuring of the surface is the average pore size of the textile fabric. The textile fabric preferably has an average pore size of 50 μm to 300 μm, preferably 70 to 250 μm, more preferably 100 to 200 μm. The pore size is measured prior to introduction into the elastomeric material. The measurement is taken in accordance with ASTM E 1294 (1989).

The bioresorbable fibers can be resorbed with time after introduction into the body. It is advantageous in this case that the bioresorbable fibers are also present within the elastomeric layer, since cavities in the layer of elastomeric material are formed during bioresorption, which is comparable to a dynamically changing, three-dimensional structuring on the surface of the soft tissue implant. Over time, the layer of elastomeric material is accordingly provided with cavities. The formation of the cavities generally takes place continuously, wherein more than half, more preferably more than 75% by weight, in particular more than 90% by weight, of the textile fabric is preferably resorbed after 60 days. As a result, the layer of elastomeric material successively becomes the surface layer of the soft tissue implant to which a permanent structuring with the aforementioned advantages can thereby be imparted. The dynamically changing surface offers, as early as during resorption, a three-dimensional environment to the endogenous cells, which environment can be populated by them and converted by normal wound healing processes. As a result, the soft tissue implant according to the invention makes it possible for body tissue to grow into it and consequently for the textile fabric to be gradually replaced by endogenous tissue.

A further advantage of the soft tissue implant according to the invention is that, at least initially after introduction into the body, the surface of the elastomeric material can be separated from the tissue in the body by the bioresorbable coating, which increases its acceptance and tissue compatibility after implantation.

Moreover, the soft tissue implant according to the invention is characterized in that it can have excellent elasticity due to the use of an elastomeric material. As a result, good adaptation to deforming forces can be ensured outside and inside the body. The high elasticity is particularly advantageous if the soft tissue implant, for example as a breast implant, is to be introduced into the body through a body opening that is as small as possible. Its high elasticity allows the soft tissue implant to deform strongly, for example to be elongated, in order to be able to be brought into the body through the small body opening.

In a preferred embodiment of the invention, the soft tissue implant is characterized by an elasticity measured in accordance with DIN 53504 S2 at a rate of 200 mm/min of 50% to 500%, preferably 200% to 500%, more preferably 400% to 500%. It was surprising for a person skilled in the art that the soft tissue implant according to the invention can have such a high elasticity. In particular, it was to be expected that delamination of the coating occurs during tensile loading. The fact that this can be avoided is probably due to the high layer adhesion of the soft tissue implant according to the invention.

The longer the time that the soft tissue implant is to remain in the human body, the stronger the advantageous effects are.

Naturally, the effects caused by the three-dimensionally structured surface come to bear the more strongly, the higher the proportion of the textile fabric in the surface of the implant is. Thus, in an advantageous embodiment of the invention, the proportion of the textile fabric in the surface of the soft tissue implant is more than 50%, more preferably more than 70%, more preferably more than 90% and in particular 100%. The aforementioned values relate to the state before introduction into the human body.

The bioresorbable fibers may comprise a wide variety of fibrous materials. The fibers preferably comprise bioresorbable fibrous materials selected from the group consisting of natural polymers, proteins, peptides, sugar, chitosan, chitin, gelatin, collagen, polyvinyl alcohol, polyvinyl pyrrolidone, dextran, pullulan, hyaluronic acid, polycapolactones, polyactides, polyglycolides, polyhydroxyalkanolates, polydioxanones, polyhydroxybutyrates, polyanhydrides, polyphosphoric esters, polyesteramides and mixtures and copolymers thereof, and/or consist of at least 70% by weight and/or at least 80% by weight and/or at least 90% by weight and/or at least 95% by weight of them, based in each case on the total weight of the bioresorbable fibers.

In a further embodiment of the invention, the fibrous material consists entirely of the aforementioned materials, wherein customary auxiliaries, for example catalyst residues, can likewise be present in the fibrous material. In a particularly preferred embodiment of the invention, the fibers exclusively have gelatin as bioresorbable fibrous material and/or consists of at least 70% by weight and/or at least 80% by weight and/or at least 90% by weight and/or at least 95% by weight of gelatin, based in each case on the total weight of the bioresorbable fibers. According to the invention, porcine gelatin is preferred, since it is not a transmitter of bovine spongiform encephalopathy (BSE). In addition, the bioresorbable fibers usually contain water. For example, in an amount of 1% by weight to 15% by weight. In a further preferred embodiment of the invention, the bioresorbable fibers additionally contain at least one hydrophilic additive. This is preferably likewise bioresorbable. Preferably, the hydrophilic additive is selected from the group consisting of: carbomer [9003-01-4], acetic acid ethenyl ester, polymer with 1-ethenyl-2-pyrrolidinone [25086-89-9], 1-ethenyl-2-pyrrolidinone homopolymer [9003-39-8], cellulose hydroxypropyl methyl ether [9004-65-3], polycarbophil [9003-97-8], 1-ethenyl-2-pyrrolidinone homopolymer [9003-39-8], methyl cellulose (E 461), ethyl cellulose (E 462), hydroxypropyl cellulose (E 463), hydroxypropyl methyl cellulose (E 464), methyl ethyl cellulose (E 465), sodium carboxymethyl cellulose (E 466), hydroxyethyl cellulose, hydroxybutyl methyl cellulose, cellulose glycolate=carboxymethyl cellulose, cellulose acetate (e.g., available from Chisso, Eastman), cellulose acetate butyrate (e.g., available from Eastman, FMC), cellulose acetate maleate, cellulose acetate phthalate (e.g., available from Eastman, FMC, Parmentier), cellulose acetate trimellitate (e.g., available from Eastman, Parmentier), cellulose fatty acid ester (cellulose dilaurate, cellulose dipalmitate, cellulose distearate, cellulose monopalmitate, cellulose monostearate, cellulose trilaurate, cellulose tripalmitate, cellulose tristearate, agar [9002-18-0], alginic acid [9005-32-7], ammonium alginate [9005-34-9], calcium alginate [9005-35-0], cellulose, carboxymethyl ether, calcium salt [9050-04-8], cellulose, carboxymethyl ether, sodium salt [9004-32-4], carrageenan [9000-07-1], carrageenan [9062-07-1], carrageenan [11114-20-8], carrageenan [9064-57-7], cellulose [9004-34-6], carob gum [9000-40-2], corn starch and pregelatinized starch, dextrin [9004-53-9], cellulose, 2-hydroxyethyl ether [9004-62-0], hydroxyethyl methyl cellulose [9032-42-2], cellulose, 2-hydroxypropyl ether [9004-64-2], cellulose, 2-hydroxypropyl ether (slightly substituted) [9004-64-2], hydroxypropyl starch [113894-92-1], ethenol, homopolymer [9002-89-5], potassium alginate [9005-36-1], sodium hyaluronate [9067-32-7], starch [9005-25-8], pregelatinized starch [9005-25-8], polyethylene oxide, polyethylene glycol. The aforementioned hydrophilic additives are present, for example, in an amount of 0.1% by weight to 30% by weight, preferably 0.5% to 20%, more preferably 1% to 10%, based in each case on the total weight of the bioresorbable fibers. According to the invention, sodium hyaluronate, hyaluronic acid, polyethylene oxide and polyethylene glycol are particularly preferred.

The advantage of using the hydrophilic additives is that they can achieve a particularly high initial wettability of, for example, less than 10 seconds, preferably less than 5 seconds, more preferably less than 2 seconds. The high initial wettability is advantageous in order to be able to impregnate the textile fabric with active ingredient solutions prior to the introduction of the soft tissue implant into the human body.

In particular with regard to the use of the soft tissue implant according to an embodiment of the invention in the human body, it may be particularly expedient if one or more medicaments selected from the group consisting of antimicrobial agents, anesthetics, anti-inflammatory agents, anti-scar agents, anti-fibrotic agents, chemotherapeutic agents and leukotriene inhibitors are present in and/or on the bioresorbable fibers. In this respect, antimicrobial substances and/or antibiotics are particularly suitable for preventing infection.

The bioresorbable fibers can be continuous filaments or staple fibers, wherein “continuous filaments” refers to fibers with a theoretically unlimited length and “staple fibers” refers to fibers with a limited length. In a preferred embodiment of the invention, the bioresorbable fibers are designed as continuous filaments and/or staple fibers with a minimum length of 5 mm, for example of 5 mm to 10 cm. In practical tests, it has been found that fibers of such length can penetrate particularly well into the layer of elastomeric material.

In a further preferred embodiment of the invention, the textile fabric has a basis weight of 10 to 300 g/m², preferably of 50 to 200 g/m², more preferably of 70 to 150 g/m². This has proven to be advantageous, since a textile fabric with such basis weights has sufficient stability in order to be able to be applied without creases to a wide variety of layers of elastomeric material of three-dimensional geometry.

Furthermore, a textile fabric with good mechanical strength can be obtained by means of the aforementioned basis weights. A maximum tensile force of at least 0.5 to 100 N, preferably of 1.0 to 50 N, more preferably of 2.0 to 30 N, can thus be imparted to the textile fabric measured with a width of 20 mm. This is advantageous, since a minimum maximum tensile force is required for processing the textile fabric.

The period of time within which the textile fabric is resorbed depends on various parameters and, among other things, also on the thickness of the textile fabric. In light of the foregoing, it has proven to be expedient in most cases to design the textile fabric with an average thickness of less than 2 mm, preferably of 5 to 700 nm.

The textile fabric can in principle comprise one or more fiber layers. It particularly preferably comprises only one fiber layer, since adhesion problems, such as often occur between a plurality of fiber layers, can be avoided.

The textile fabric can also be present in a wide variety of embodiments, for example as woven fabric, knitted fabric or non-woven fabric. Non-woven fabrics, as described above, are particularly preferred according to the invention, in particular non-woven fabrics produced in a rotary spinning process. In rotary spinning processes, non-woven fabrics can be produced, for example, by providing a fluid which contains fibrous material and which can be present as a melt, solution, dispersion or suspension, spinning the fluid by rotary spinning, stretching it and depositing it into a non-woven fabric. With this technique, work can be carried out at low temperatures up to 60° C. This enables particularly gentle processing of the biopolymers and active ingredients.

Non-woven fabrics particularly preferred according to the invention are non-woven fabrics as described in WO 2008/107126 A1, WO 2009/036958 A1, EP 2 409 718 A1, EP 2 042 199 A1, EP2129339B1, CA2682190C. The aforementioned publications are incorporated by reference herein.

The layer of elastomeric material may have a wide variety of elastomeric materials. Of these materials, silicone elastomers, especially medical-grade silicone elastomers, are particularly preferred, because they are relatively inert and do not react with the body. Preferably, the layer of elastomeric material is at least 70% by weight and/or at least 90% by weight and/or at least 95% by weight of the aforementioned silicone elastomers. Most preferably, the layer of elastomeric material consists of 100% by weight of medical-grade silicone elastomers, wherein customary additives may be contained.

The thickness of the layer comprising the elastomeric material may vary depending on the materials used and the intended use. Thicknesses in the range of 100 μm to 5000 μm, preferably of 100 μm to 4000 μm, more preferably of 100 μm to 3000 μm, have proven to be generally favorable. In principle, the layer of elastomeric material may comprise one or more layers. In one embodiment of the invention, the soft tissue implant has a backing layer. This backing layer is preferably arranged on the side, facing away from the textile fabric, of the layer comprising the elastomeric material. The backing layer preferably consists of a biocompatible material, since it can remain in the soft tissue implant and meets the requirements for introduction into the human body. For this reason, the backing layer preferably consists of an elastomeric material, in particular of silicone. The use of other backing layers, for example foils, plates or molded bodies, is also conceivable.

An embodiment of the present invention also relates to forming the soft tissue implant as a breast implant. An implant is understood to mean a material which is implanted into the body and is intended to remain there permanently or at least for a period of time, for example of a few days to 10 years.

In a particularly preferred embodiment of the invention, the soft tissue implant is designed as a breast implant and has the following features:
The layer of elastomeric material is in the form of a bubble-like shell.
The shell can be filled and/or is filled with a liquid to viscous filling material.
The textile fabric having bioresorbable fibers is arranged as a coating on the outside of the shell.

In the case of implants of this type, the entire surface can be formed by the coating, such that the aforementioned advantages can be utilized particularly efficiently. According to the invention, the coating thus preferably completely covers the outside of the shell in this embodiment.

The soft tissue implant is expediently shaped in such a way that it can fill a cavity in the human body according to shape and size.

In a preferred embodiment of the invention, the soft tissue implant according to the invention can be produced by a method comprising the following steps:
1. providing a backing layer;
2. applying a biocompatible elastomeric precursor material, in particular of unvulcanized silicone, to one side of the backing layer;
3. applying a textile fabric having bioresorbable fibers to the elastomeric precursor material, such that the fibers of the textile fabric at least partially penetrate into the elastomeric precursor material;
4. crosslinking the elastomeric precursor material into an elastomeric material.

The first method step comprises providing a backing layer. A biocompatible material is preferably used as the backing layer, since it can remain in the soft tissue implant and meets the requirements for introduction into the human body. For this reason, the backing layer preferably consists of an elastomeric material, in particular of silicone. The use of other backing layers, for example foils or molded bodies, is also conceivable.

The second method step comprises applying a biocompatible elastomeric precursor material, in particular of unvulcanized silicone, to one side of the backing layer. A wide variety of materials, such as unvulcanized and/or not completely vulcanized silicone, can be used as elastomeric precursor material. Such materials can be converted into elastomeric materials by crosslinking in the form of vulcanization. When using silicone in the backing layer and an elastomeric silicone precursor material, it is advantageous that a particularly homogeneous connection is formed between the layers, since both layers then have the same properties.

The third method step comprises applying a textile fabric having bioresorbable fibers to the elastomeric precursor material, such that the fibers of the textile fabric at least partially penetrate into the elastomeric precursor material. The penetration of the fibers of the textile fabric into the elastomeric precursor material can be accomplished, for example, by pressurizing the composite of textile fabric and elastomeric precursor material. For this purpose, the elastomeric precursor material preferably has a viscosity of 200 mPa*s to 4000 mPa*s, more preferably of 300 mPa*s to 3000 mPa*s and in particular of 500 mPa*s to 2000 mPa*s. The textile fabrics mentioned above are preferably used as textile fabric. Particular preference is given here to fabrics comprising fibers made of gelatin.

The fourth method step comprises crosslinking the elastomeric precursor material into an elastomeric material. When silicone precursor materials are used, crosslinking can be carried out easily by heating (vulcanization). It was surprising for a person skilled in the art that crosslinking also works in the presence of a textile fabric containing gelatin, since gelatin is known to have a multitude of functional groups. The latter are known to the person skilled in the art as catalyst poison.

It is conceivable to remove the backing layer after the crosslinking step. However, when a biocompatible backing layer is used, it is preferred if it remains in the soft tissue implant.

As already explained above, the soft tissue implant according to the invention is outstandingly suitable in an embodiment as a breast implant.

In a further embodiment of the invention, it also comprises the breast implant itself. In its embodiment as a breast implant, it preferably has a bubble-like shell of elastomeric material, in particular of silicone, as a layer comprising an elastomeric material, wherein the shell can be filled and/or is filled with a liquid to viscous filling material. If a shell which has not yet been filled with filling material is used, it can be filled and subsequently closed easily even after its connection to the textile fabric.

The filling material may comprise a wide variety of materials. The filling material is preferably selected from the group consisting of saline solution, viscous saline solution, silicone gel, hydrogel and thermoreversible polymer gel.

On the outside of the shell, the implant has a textile fabric having bioresorbable fibers, wherein the bioresorbable fibers at least partially penetrate the shell of elastomeric material. The breast implant is expediently shaped in such a way that it can fill a cavity in the human body according to shape and size.

In summary, the soft tissue implant according to the invention can be produced, for example, by a method comprising the following steps:
1. providing a bubble-like shell of elastomeric material, wherein the shell can be filled and/or is filled with a liquid to viscous filling material;
2. applying an elastomeric precursor material, in particular unvulcanized silicone, to the outside of the shell;
3. applying a textile fabric having bioresorbable fibers to the outside of the shell;
4. treating the composite of textile fabric and shell with pressure, for example, such that the fibers of the textile fabric penetrate at least partially into the shell;
5. crosslinking the material.

Embodiments of the present invention are explained in more detail below with reference to an example.

### Example: Production of a Soft Tissue Implant According to an Embodiment of the Invention

The following starting materials are used for producing an elastomeric precursor material: MED-6400A (component A) and MED-6400B (component B) NuSil Technology. Components A and B are mixed with a weight ratio of 1:1 at room temperature. The mixture is further processed without bubbles. The elastomeric precursor material obtained in the process is cast onto the surface of a bubble-like shell as backing layer. The shell coated with elastomeric precursor material is held horizontally for 30 minutes for leveling and evaporation of the solvent. A gelatin non-woven fabric is subsequently placed onto the surface of the coated shell. The composite of gelatin non-woven fabric and silicone-coated shell is produced by crosslinking the elastomeric precursor. For this purpose, treatment is carried out in a programmable oven with the following temperature program: 30 minutes at room temperature, 45 minutes at 75° C. and 135 minutes at 150° C. with constant change (programming). After the cooling of the hardened sample, a silicone/gelatin composite non-woven fabric is obtained. Tensile tests for the obtained soft tissue implant according to the invention are carried out with a tensile testing machine in accordance with DIN 53504 S2 at a head speed of 200 mm/min.

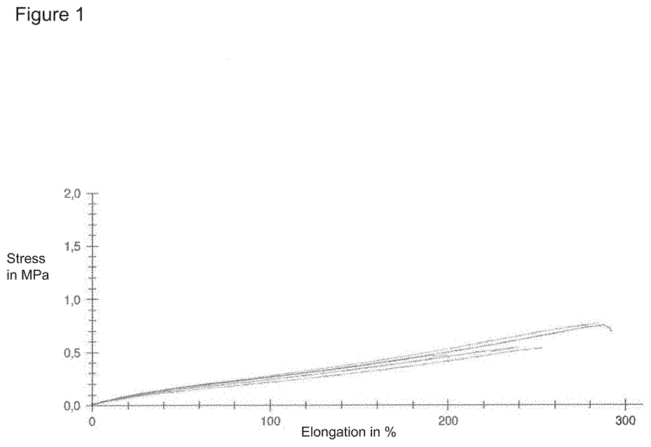

FIG. 1 shows as a reference the result of a tensile test with a pure silicone layer. A linear progression of the tensile stress curve typical of elastomers is evident. With maximum stresses of 0.4 MPa to 0.7 MPa at a maximum elongation of 200% to 300%.

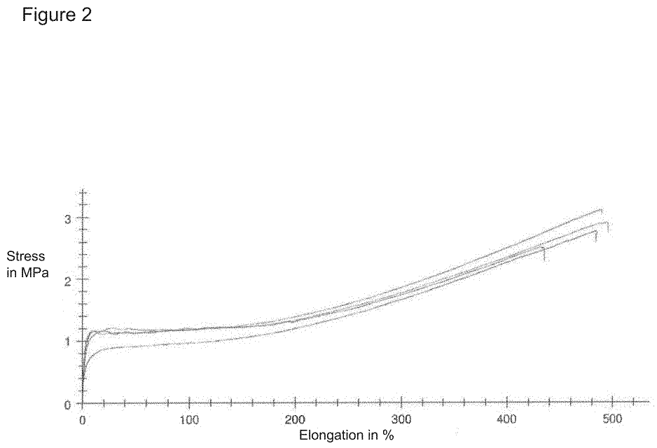

FIG. 2 shows the result of a tensile test of the soft tissue implant of Example 1. The gelatin non-woven fabric in the composite brings about a high stress absorption of approximately 1 MPa with a low elongation of 10-20%. The maximum elongation (HZD) is almost doubled at 400%-500% compared to the pure silicone layer, presumably because the fibers break independently of the elastomer and hold it together longer. Starting at 200% elongation, the stress absorption again increases linearly. In this range, the elastomer portion presumably absorbs the applied forces, while the forces were previously absorbed by the non-woven fabric up to 200% elongation. The maximum tensile force (HZK) in this composite at 2.4 to 3 MPa is five times higher than in a pure silicone layer.

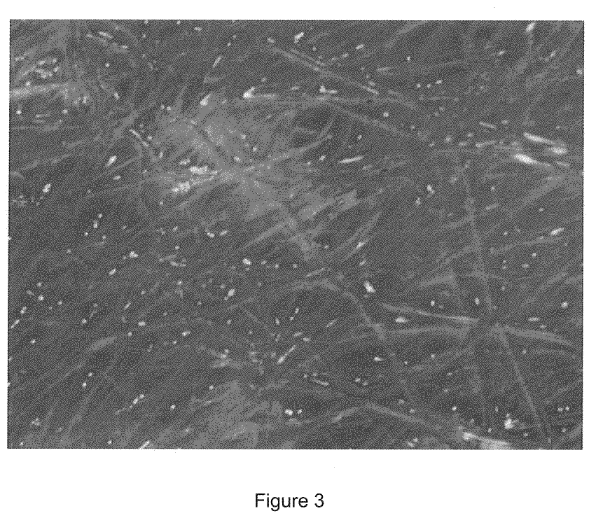

FIG. 3 shows a microscopic image of the surface of the soft tissue implant of Example 1 after two weeks of storage at 37° C. in PBS. The crosslinked fibers are still visible at this time.

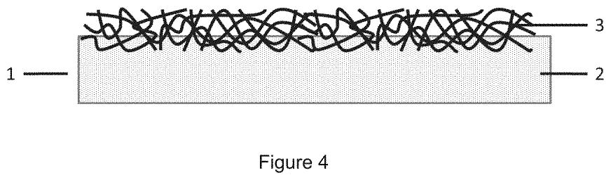

FIG. 4 shows the schematic cross-section of a soft tissue implant (1) according to the invention comprising a layer (2) of an elastomeric material, and a textile fabric (3) arranged on this layer (2) and forming the surface of the soft tissue implant, wherein the textile fabric (3) has bioresorbable fibers which are at least partially embedded in the layer (2) of elastomeric material.

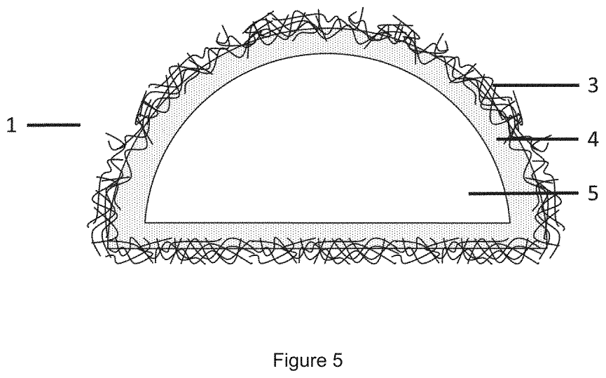

FIG. 5 shows the schematic cross-section of a soft tissue implant (1) according to the invention in its embodiment as a breast implant. The breast implant has a bubble-like shell (4) made of elastomeric material, here silicone, wherein the shell (4) is filled with a liquid to viscous filling material (5). On the outside of the shell (4), the implant has a textile fabric (3) having bioresorbable fibers, wherein the bioresorbable fibers at least partially penetrate the shell (4) of elastomeric material.

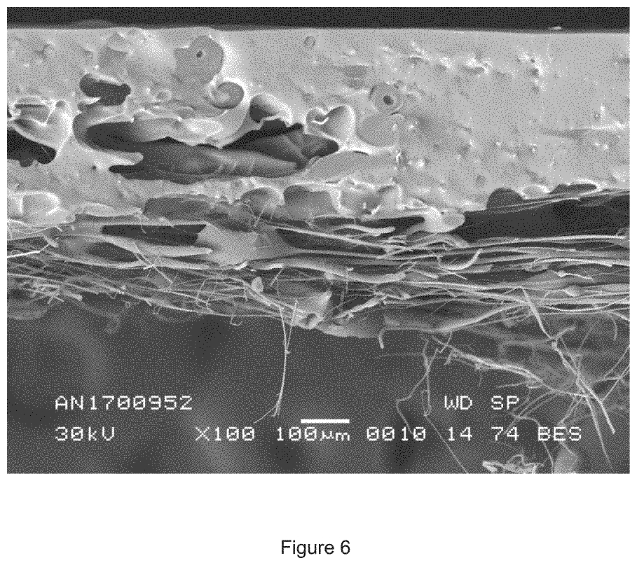

FIG. 6 shows an electron micrograph of the sectional view of a soft tissue implant according to the invention. A gelatin non-woven fabric is arranged as textile fabric on the layer of elastomeric material, here silicone. It can be clearly seen how the fibers of the gelatin non-woven fabric penetrate into the layer of silicone.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A biocompatible soft tissue implant for introduction into a human body, the biocompatible soft tissue implant comprising:
at least one layer comprising an elastomeric material; and
at least one textile fabric arranged on the at least one layer comprising the elastomeric material and forming a surface of the biocompatible soft tissue implant,
wherein the at least one textile fabric includes bioresorbable fibers which are embedded at least partially embedded in the at least one layer comprising the elastomeric material.

2. The biocompatible soft tissue implant according to claim 1, wherein the embedding of the bioresorbable fibers into the at least one layer comprising the elastomeric material was obtained by applying the at least one textile fabric to an elastomeric precursor material and pressing the at least one textile fabric into the elastomeric precursor material.

3. The biocompatible soft tissue implant according to claim 1, wherein the at least one textile fabric is a non-woven fabric.

4. The biocompatible soft tissue implant according to claim 1, wherein the at least one textile fabric has an average pore size of 50 μm to 300 μm.

5. The biocompatible soft tissue implant according to claim 1, wherein after introduction into the human body, cavities are formed in the at least one layer comprising the elastomeric material over time due to the bioresorption of the at least one textile fabric.

6. The biocompatible soft tissue implant according to claim 1, having an elasticity measured in accordance with DIN 53504 S2 at a rate of 200 mm/min of 50% to 500%.

7. The biocompatible soft tissue implant according to claim 1, wherein a proportion of the textile fabric at the surface of the soft tissue implant is more than 50%.

8. The biocompatible soft tissue implant according to claim 1, wherein the bioresorbable fibers have bioresorbable fibrous materials selected from the group consisting of natural polymers, proteins, peptides, sugar, chitosan, chitin, gelatin, collagen, polyvinyl alcohol, polyvinyl pyrrolidone, dextran, pullulan, hyaluronic acid, polycapolactones, polyactides, polyglycolides, polyhydroxyalkanolates, polydioxanones, polyhydroxybutyrates, polyanhydrides, polyphosphoric esters, polyesteramides and mixtures and copolymers thereof, and/or consist of at least 70% by weight and/or at least 80% by weight and/or at least 90% by weight and/or at least 95% by weight of them, based in each case on the total weight of the bioresorbable fibers.

9. The biocompatible soft tissue implant according to claim 1, wherein one or more medicaments selected from the group consisting of antimicrobial agents, anesthetics, anti-inflammatory agents, anti-scar agents, anti-fibrotic agents, chemotherapeutic agents and leukotriene inhibitors are present in and/or on the bioresorbable fibers.

10. The biocompatible soft tissue implant according to claim 1, wherein the bioresorbable fibers are designed as continuous filaments and/or staple fibers with a minimum length of 5 mm.

11. The biocompatible soft tissue implant according to claim 1, wherein the at least one textile fabric is a non-woven fabric produced in a rotary spinning process.

12. The biocompatible soft tissue implant according to claim 1, wherein the at least one layer comprising the elastomeric material has silicone elastomers.

13. The biocompatible soft tissue implant according to claim 1, configured as a breast implant for implantation into the human body having the following features:
a. the at least one layer comprising the elastomeric material is in a form of a bubble-like shell,
b. the shell can be filled and/or is filled with a liquid to viscous filling material, and
c. the at least one textile fabric having bioresorbable fibers is arranged as a coating on an outside of the shell.

14. A method for producing a biocompatible soft tissue implant according to claim 1, the method comprising:
a. providing a backing layer;
b. applying a biocompatible elastomeric precursor material to one side of the backing layer;
c. applying the at least one textile fabric having the bioresorbable fibers to the elastomeric precursor material such that the fibers of the textile fabric at least partially penetrate into the elastomeric precursor material; and
d. crosslinking the elastomeric precursor material to form the elastomeric material.

15. The method according to claim 14, wherein the biocompatible elastomeric precursor material comprises an unvulcanized silicone.

16. The biocompatible composite material according to claim 2, wherein the biocompatible elastomeric precursor material comprises an unvulcanized silicone layer.

17. The biocompatible composite material according to claim 1, wherein the at least one textile fabric has an average pore size of 100 μm to 200 μm.

18. The biocompatible composite material according to claim 1, having an elasticity measured in accordance with DIN 53504 S2 at a rate of 200 mm/min of 400% to 500%.

19. The biocompatible composite material according to claim 1, wherein a proportion of the textile fabric at the surface of the soft tissue implant is approximately 100%.

20. The biocompatible composite material according to claim 1, wherein the bioresorbable fibers are configured as continuous filaments and/or staple fibers with a length of 5 mm to 10 cm.
